(19) United States
(12) Patent Application Publication
(10) Pub. No.: US 2009/0312582 A1
(43) Pub. Date: Dec. 17, 2009

(54) Synthesis of butylated hydroxyanisole from tertiary butyl hydroquinone

(75) Inventors: Mangesh Narayan Rajadhyaksha, Maharashtra (IN); Arun Kumar Sharma, Maharashtra (IN); Rajesh Shriram Gupta, Maharashtra (IN); Ajit Shripad Shirwaikar, Maharashtra (IN)

Correspondence Address: D. PETER HOCHBERG CO. L.P.A., 1940 EAST 6TH STREET, CLEVELAND, OH, 44114, US

(73) Assignee: Camlin Fine Chemicals Limited, Maharashtra (IN)

(21) Appl. No.: 11/918,838

(22) PCT Filed: Apr. 13, 2006
(86) PCT No.: PCT/IN2006/000132

§ 371 Date: Oct. 19, 2007

(30) Foreign Application Priority Data
Apr. 19, 2005 (IN) 489/MUM/2005

(51) Int. Cl. C07C 43/23; C07C 41/01; C07C 41/40; C07C 41/34

(52) U.S. Cl. 568/650

(57) ABSTRACT

A process for production and novel physical forms of butylated hydroxyanisole (BHA) wherein tertiary butyl hydroquinone (TBHQ) reacts within a range of about 30 to 50° C. with stoichiometrically slight excess of dimethyl sulphate and sodium hydroxide. The sodium hydroxide is added stoichiometrially in slight excess of dimethyl sulphate. A major portion of BHA formed in this reaction was recovered in a crystalline form in which 3-t-butyl-4-hydroxyanisole (3 isomer) is at least at a level of about 99%, usually at about 99.5% or more, and TBHQ at 100 ppm or less. BHA remaining in a mother liquor after crystallization was recovered by distillation and had the same purity as the crystalline BHA. The crystalline form was prepared as a low density, as well as a high density, form and could also be converted into compressed forms including flakes, tablets and the like.

### CROSS-REFERENCE TO RELATED APPLICATIONS

This application is a National Stage application of International Application No. PCT/IN2006/000132, filed on Apr. 13, 2006, which claims priority of Indian patent application number 489/MUM/2005, filed on Apr. 19, 2005, both of which are incorporated by reference in their entiries herein.

### BACKGROUND OF THE INVENTION

1. Field of the Invention

The present invention relates to a process for synthesis of butylated hydroxyanisole (BHA) and novel physical forms of BHA.

2. Description of the Prior Art

Butylated-hydroxyanisole (BHA) also known as 3-tertiary-butyl- 4-hydroxyanisole, having two isomers, is widely used as an antioxidant for fats and oils and finds extensive usage in food, pharma, petroleum and cosmaceutical industry. The commercial BHA is available as a mixture of 3-t-butyl-4-hydroxyanisole (the 3 isomer) which is in a dominant proportion and 2-t-butyl-4-hydroxyanisole (2 isomer). The 3 isomer is preferred for its antioxidant property.

The prior art patent documents teach us various methods for synthesis of BHA.

U.S. Pat. No. 4,538,002 has described a process for the production of alkylated hydroxyanisoles consisting of dehydrogenating para-isopropylphenol to para-isopropenylphenol which is further reacted with a methylating agent to yield para-isopropenylanisole followed by treatment with acidic hydrogen peroxide to yield para-hydroxyanisole which is then treated with an alkylating agent to yield the alkylated hydroxyanisole. The process of this invention is especially useful for the synthesis of the antioxidant butylated hydroxyanisole.

U.K. patent No. GB 1366441 also describes one such method for synthesis of BHA. The method essentially involves the reaction of TBHQ (t-butyl-hydroquinone), heptane, water at 30° C. to 50° C. to which is further added dimethyl sulphate (DMS) and aqueous sodium hydroxide solution over a period of time. The reaction mixture is further heated rapidly to 40-42° C. The mixture was stirred for another 15 minutes at 35° C. to 40° C., water was added and the mixture was heated rapidly to 70° C. to destroy any unreacted dimethyl sulphate. The aqueous layer was drained off and the organic layer was washed five times with hot water containing sodium hydroxide followed by aqueous acetic acid and finally with hot water. The organic layer was then stirred to give a material containing 5% t-butyl-1,3-dimethoxybenzene and 86.5% BHA, and 82.3% yield. Recovery of BHA containing material from the organic layer was done by distilling out hexane at pot temperature of 130° C. The BHA contained 96.8% by weight of the 3-isomer. In this process there is claimed use of a molar excess of dimethyl sulphate as well as TBHQ over sodium hydroxide. Total quantity of water used in this process is from 2.5 to 5 parts by weight per 10 parts by weight of dimethyl sulphate.

In U.S. Pat. No. 2,887,515, Young and Rodgers (1959) described a method for preparation of Tertiary Butyl-4-Methoxy phenol wherein a major portion of 2-tertiary butyl-4-methoxyphenol and a minor proportion of 3-tertiry butyl-4-methoxy phenol which comprises reacting mono-tertiary butyl hydroquinone with dimethyl sulfate, methyl chloride or methyl acid sulfate in an alkaline aqueous solution containing zinc dust at an elevated temperature.

In U.S. Pat. No. 2,776,321, Clemens has claimed a process where conversion of more than 90% of monotertiarybutyl hydroquinone to the monoether mixture is reported. However, nearly one third of the product is the relatively low potency 3-tertiarybutyl isomer.

U.S. Pat. No. 2,801,268 describes work of Brimer and Kingsport wherein a method is disclosed to achieve minimum amounts of 3-tertiarybutyl isomer of lower antioxidant activity and recover a final product which contained about 5% of the 3-tertiarybutyl isomer.

In all of the above processes, conversion efficiency from TBHQ to the product is far less than 100%, impurities are formed and the product at the end needs to be recovered free from TBHQ either by alkali extraction or distillation. Further, in all prior art processes, level of the 2 isomer, which is an unwanted isomer, is more than 1% in the final product. In none of the prior art processes isolated BHA has more than 99% purity of required 3 isomer.

### SUMMARY OF THE PRESENT INVENTION

This invention covers an improved process for synthesis and one or more of a novel physical forms of BHA.

The invented process comprises the use of slight excess of dimethyl suplhate than TBHQ in an agitated reaction carried out in hexane at 30° C. to 50° C. to which sodium hydroxide is added, which is in stoichiometrically slight excess in quantity to dimethyl sulphate. The quantity of sodium hydroxide used is such that it is just enough to give a slightly alkaline pH after the reaction is over and it is added over a period of time, stirred further at about 25 to 30° C. for a period of time, then cooled to about 20-25° C., adjusted to pH 3 to 4 using acid, stirred further at 25-30° C. for a period of time and allowed to settle. Total quantity of water used in this reaction was about 3.3 parts by weight or more per 10 parts by weight of dimethyl sulphate used. Surprisingly practically complete conversion of TBHQ is achieved and less than 100 parts per million (ppm) of TBHQ remains in the final product. In rare cases when TBHQ remains unreacted at 0.5% or above, a wash with 0.5% or 1% alkali is given to the organic layer. About 70% of BHA from this organic layer can be recovered very easily as a crystalline lump by simply lowering the temperature below 10° C. to −10° C., sedimentation of the crystals, draining off of the mother liquor. These crystals have 3 isomer at 99.5% or more and TBHQ maximum at 100 ppm, making this product usable without any further purification step.

The crystals, for better handling, can be converted to novel physical forms including flakes under agitated filtration under pressure or compressed into tablets or similar compressed form.

BHA remaining in mother liquor is recovered by distillation followed by the lowering of the temperature of the recovered BHA and crystallization. This BHA has 99% of 3 isomer in it.

### DETAILED DESCRIPTION OF THE INVENTION

One embodiment of this invention comprises the preparation of BHA by reacting TBHQ with a dialkyl etherifying agent in the presence of NaOH, wherein TBHQ is taken is in a slightly less quantity stoichiometrically than that of the dialkyl etherifying agents, such as dimethyl sulphate, and the dimethyl sulphate itself is also taken in a slightly less quantity stoichiometrically than that of NaOH. The extent of the slight excess of NaOH is required to be in a quantity which is enough to ensure that the pH of the reaction mixture at the end of reaction/NaOH addition is slightly alkaline. The reaction is done at 30-50° C. over a period of time.

The result of this step is the next embodiment of this invention wherein in the process of this invention, TBHQ added in the reaction described in the preceding paragraph is utilized very close to 100%, less than 0.01% to 0.03% of TBHQ remains in the organic solvent layer after its water washing, which does not need removal by fractional distillation and can be allowed to come into the product without exceeding concentration of about 100 ppm TBHQ in the final recovered product.

It is also an embodiment of this invention that a simple step of reducing temperature of a process stream containing BHA, one such process stream comprising the organic solvent layer obtained after completion of etherification reaction, to around 10° C. or less up to −100° C. leads to crystallizing out of 70% of the BHA formed as a pure product in a solid lump or as a slurry with less than 100 ppm TBHQ in it which can be easily separated from the rest of the reaction mixture by a simple process of separation of solid from liquids comprising one or more of a filtration, a sedimentation, a centrifugation and the like. This process is far more simple, convenient and cost efficient as compared to the prior art process where all of the BHA formed as a result of etherification reaction is recovered by distillation of the entire reaction mixture. In the process of this invention, about 70% of the BHA formed is recovered as crystals formed by lowering of temperature below 10° C. and up to −10° C. and to that extent, load on distillation process is reduced. This has made the process of production highly simplified and cost effective too. Of course, even after practicing other embodiments of this invention, one may choose to isolate BHA by fractional distillation exclusively without resorting to crystallization for any reason, and that method is also an embodiment of this invention and included within the scope of this invention.

It is a further embodiment of this invention that the crystallized out product has less than 0.5% of 2 isomer in it.

In a further embodiment of this invention, a part of the BHA remaining in the mother liquor after crystallization of major portion of the BHA is recovered by fractional distillation under vacuum. This BHA has minimum of 99% of the 3 isomer. In prior art processes, a level of isomer more than 95% has been claimed, however a composition having 99% or more of 3 isomer was never achieved.

It was found that the crystalline BHA of this invention formed by reduction in temperature exhibited a few problems in handling and use. Crystallized BHA had a very low bulk density and was dusty in nature. Further, this material being light, was also taking a long time to dissolve because it remained floating on the oil surface and being a fine crystalline material, used to give a recrystallizing effect in the oil solution. These problems were eliminated in an embodiment of this invention by a process of filtration of slurry of these crystals under pressure accompanied by agitation achieved preferably by using agitated Nutsche filter, followed by vacuum drying. This resulted into formation of a product of a better physical form which had a higher bulk density and less of a dusting effect, also dissolved very easily at ordinarily warm temperature in oils and did not give a recrystallizing effect.

This high density product, however, had a tendency of agglomeration into a hard rock like mass after some time in storage, which presented inconvenience in usage. In the next embodiment of this invention, which produced newer physical forms, this product, before agglomeration, was melted and flaked or compacted as round or hexagonal shaped tablets and the like. The physical form of flakes or compacted tablet have provided a decided technical advantage in handling and usage of BHA.

It is an important embodiment of this invention, that solvent is added only once in the reaction mixture and the conversion of TBHQ is practically complete. This has made this process very simple to operate and the proportion of 3-isomer in the solidified crystalline lump is about 99.5% or more.

The composition of BHA of this invention prepared by crystallization induced by lowering of temperature of the process flow solution to lower than 10 degrees contains 99.5% as the desired isomer and impurities remain in the filtrate. The pure form of the product has less than 100 ppm of the unreacted TBHQ.

The process is far less cumbersome compared to the prior art method as it does not need distillation to remove the impurities and other reactants.

Specific examples are given in the following which are for the purpose of illustrating working and various embodiments of this invention without limiting the scope of this invention to the reactants and exact reaction conditions used and anything that is equivalent within the scope of the claims of this invention or an adaptation obvious to an ordinary person skilled in the art is included as an embodiment of this invention.

Unless context indicates otherwise, a mention of singular also includes plural. Thus, “a hydroxide” includes one or more of all known hydroxides; “a alkali metal” includes one or more of all known alkali metals and the like.

### EXAMPLE 1

In a 5 L four necked round bottom flask fitted with a stirrer, thermometer pocket and dropping funnel was placed 166 g TBHQ suspended in 83 ml water and 996 ml Hexane and stirred at 25-30° C. for 15-20 minutes. To this was added 159.32 g di-methyl sulphate and was further stirred for 15 minutes. A solution of 55.6 g sodium hydroxide in 111 ml water was placed in a dropping funnel and was added to the reaction mixture drop-wise maintaining the reaction temperature below 45-50° C. over a period of 1 to 4.5 hours. After the addition is over, the reaction mixture was further stirred at 25-30° C. for one hour. The reaction is monitored by thin layer chromatography. The reaction mixture is then cooled to 20-25° C. and adjusted to a pH of 3 to 4 with 50% sulphuric acid and stirred further at 25-30° C. for 15-20 minutes. It was then allowed to settle for 30 minutes.

Aqueous layer was separated and discarded. Hexane layer was washed with 250 ml water and further it was optionally washed with sodium hydroxide solution, preferably by a 0.5% to 1% solution of sodium hydroxide, in case TBHQ and other impurities are detected in process sample in undesired proportion such as around 0.5% of TBHQ and around 0.6% of other impurity such as self oxidation product of BHA. It was then filtered through HYFLO® bed. The HYFLO® bed was washed with 100 ml hexane. The total hexane layer was cooled to 0 to 5° C. and maintained at this temperature for 1 to 2 hours. The solid separated was filtered and washed with 260 ml chilled hexane. The solid filtered was sucked dry, and dried in a tray drier at 40° C. till constant weight. Weight of the dry product obtained was 116 g having M.P. 58-61° C.

### EXAMPLE 2

### Scale Up

Experiment 1 was repeated at scale of batches involving use of 1000 kg or more of TBHQ per batch and results obtained by varying relative stoichiometric ratios of TBHQ, DMS and NaOH, which included ratios, respectively, of (1:1.1: 1.33), (1:1.1:1.46), (1:1.26:1.38), (1:1.1.4:1.46), (1:1.26:1,4), (1:1.1:1.2) and (1:1.03:1.2). Out of these ratios (1:1.26:1.38), (1:1.1.4:1.46) were considered as close to ideal in practically 100% or very close to 100% conversion of TBHQ as well as high yield of BHA having purity of 99.5% or more for the 3-isomer; and (1:1.26:1.38) was found to be practically preferable. Of course, any other stoichiometric ratios of TBHQ, DMS and NaOH where DMS used is slightly in excess of TBHQ and at the same time, NaOH is in slight excess of DMS can be resorted to and they shall still be within the scope of this invention, although above ratios have been determined as preferred ones.

### EXAMPLE 3

### Different Physical Forms of BHA

From large scale production following the stoichiometric ratios described in Example 1, by cooling the final hexane layer, a slurry was obtained in which BHA separated in a crystalline form.

The crystals were separated by passing this slurry through agitated Nutsche filter and the crystalline solid thus separated.

This crystalline form led to formation of two more physical forms, a flake form and a compressed form.

A flake form was formed when the crystals were melted by passing steam through the jacket and the molten form of BHA is passed through a flaker machine having cold water circulation. After cooling, BHA got solidified in that treatment into a form of flakes.

Compressed form, such as tablets of various sizes were formed when the crystalline BHA separated over the Nutsche filter was subjected to the tabletting machines commonly known in this field of art and tablets of various sizes were formed.

### EXAMPLE 4

Recovery of BHA from Mother Liquor

Hexane from mother liquor obtained from Example 1 was distilled under vacuum (about 60 mm) at a temperature from 60-90° C. The dark red brown oil obtained was degassed and then taken for high vacuum distillation (0.5-2 mm). This residual oil on gas chromatography shows 42-52% of methyl ether of TBHQ, 41 to 46% of required BHA isomer and around 4 to 7% unwanted BHA isomer. In this experiment, 1.77 Kg of the degassed oil was distilled under vacuum and three different fractions were collected. The results obtained are given in Table 1.

TABLE 1
Fractional distillation of mother liquor for recovery of BHA

| | | | | | | G.C. ANALYSIS | |
|---|---|---|---|---|---|---|---|
| | KETTLE | VAPOUR | | | QUANTITY | METHYL ETHER | TOTAL BHA |
| FRACTION | TEMP | TEMP | VACUUM | | DISTILLED | OF TBHQ (approx) | (approx) |
| 1 | 122 to 130° C. | 55 to 115° C. | 1 | mm | 0.770 kg | 85.00% | 4 to 5% |
| 2 | 130 to 136° C. | 116 to 126° C. | 1 to 1.2 | mm | 0.251 kg | 60.00% | 40.00% |
| 3 | 137 to 165° C. | 127 to 129° C. | 1.3 | mm | 0.535 kg | 1.00% | 85 to 97% |

The third fraction containing 85-97% of BHA (0.535 Kg) was dissolved in hexane (1.5 lit) at 50 to 55° C. and the clear solution was cooled at 0-5° C. and maintained at this temperature for 2 hours. The separated solid was filtered, washed with 2×100 ml. chilled hexane and the solid was air dried in a tray drier at 40° C. The purity of this BHA was found to be Minimum 99% of required isomer and dry weight 0.461 Kg.

Hexane is recovered by distillation and can be reused.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. A process for preparing butylated-hydroxyanisole (BHA) wherein aqueous solution of a hydroxide of an alkali metal added over a time from about 1 to 4 hours to an agitated system of tertiary butyl hydroquinone (TBHQ) and a dialkyl etherifying agent in an organic hydrocarbon solvent at a temperature ranging from 25 to 50° C. more preferably below 45° C. wherein:
a. the said hydroxide is taken stoichiometrically in slight excess of the dialkyl etherifying agent,
b. the said dialkyl etherifying agent is taken stoichiometrically in slight excess of TBHQ and
c. total quantity of water used in the reaction is around 3 or more parts by weight per 10 parts by weight of dimethyl sulphate.

2. A process of claim 1 wherein,
a. the said hydrocarbon solvent comprises of one or more of hexane, toluene, pentane, heptane, and like
b. the hydroxide of the said alkali metal comprises of one or more of hydroxide of lithium, sodium, potassium, rubidium or cesium, preferably of sodium hydroxide;
c. the said dialkyl etherifying agent comprises of one or more of, dimethyl sulphate and diethyl sulphate preferably dimethyl sulphate.

3. A process of claim 1 wherein, for every mole of TBHQ suspended in about 85 ml water,
a. the said organic solvent comprising hexane is taken in around 1000 ml,
b. the said hydroxide of alkali metal comprising sodium hydroxide is taken at around 1.26 moles,
c. the said dialkyl etherifying agent comprising dimethyl sulphate is taken at around 1.39 moles,
d. the total quantity of water in the reaction mixture used is around 190-220 ml preferably 210 ml.

4. A process wherein,
a. optionally the reaction mixture formed at the end of the process of claim 3 is stirred further at around 25-30° C. for about one hour to ensure completion of the reaction,
b. the reaction mixture is adjusted to pH of about 3 to 4 with an acid, preferably 50% sulphuric acid taking care to avoid temperature going above around 40° C., preferably by cooling the reaction mixture to about 20-25° C. before addition of acid,
c. the reaction mixture is optionally stirred further at about 25 to 30° C. for 15-20 minutes, and allowed to settle for about 30 minutes, d. the hexane layer is optionally washed with dilute alkali solution, preferably by solution of sodium hydroxide at around 0.5 to 1%,
e. the hexane layer is processed further for recovery of BHA as well as recovery of hexane itself.

5. A process of recovering BHA from a process stream by cooling the same to below 10° C., preferably to about 5° C. and maintaining the same at that temperature preferably for about 1 to 2 hours, where the said process stream comprises one or more of a reaction mixture of claim 4, a dark brown oil of BHA recovered from distillation of a reaction mixture, a solution of BHA done for further purification of BHA and the like, to get two phases,
a. one phase being major part of BHA crystallized out in solid form, and
b. the other phase being a Mother Liquor, which is the residual liquid reaction mixture,
c. the two phases being capable of separating from each other by a process of separation of solids from liquids including one or more of sedimentation and decantation, filtration, filtration under pressure through appropriate equipment preferably a Nutsche filter, centrifugation; and
d. the separated crystalline composition of BHA is optionally washed with a liquid, preferably chilled hexane.

6. Crystalline BHA prepared by process of one or more of claim 1, claim 2, claim 3, claim 4 wherein 3-t-butyl-4-hydroxyanisole (3 isomer) is present at least at about 99% level, usually at about 99.5% or more.

7. Crystalline BHA prepared by process of one or more of claim 1, claim 2, claim 3, claim 4 wherein TBHQ is present maximum at 100 parts per million level.

8. Crystalline BHA wherein 3-t-butyl-4-hydroxyanisole (3 isomer) is present at least at about 99% level, usually at about 99.5% or more.

9. Crystalline BHA prepared by process of one or more of claim 1, claim 2, claim 3, claim 4 wherein TBHQ is present maximum at 100 parts per million level and 3-t-butyl-4-hydroxyanisole (3 isomer) is present at least at about 99% level, usually at about 99.5% or more.

10. Crystalline BHA wherein TBHQ is present maximum at 100 parts per million level and 3-t-butyl-4-hydroxyanisole (3 isomer) is present at least at about 99% level, usually at about 99.5% or more.

11. A process of claim 5 wherein the Mother Liquor is subjected to:
a. distillation under high vacuum over a period of time, preferably for one hour, at an elevated temperature, preferably to about 60 to 90° C. to get a dark red brown oil,
b. the dark red brown oil being degassed if required and subjected to high vacuum distillation at about 0.5 mm to 2 mm to get a fraction rich in BHA.

12. Crystalline BHA prepared by process of claim 11 wherein TBHQ is present maximum at 100 parts per million level and 3-t-butyl-4-hydroxyanisole (3 isomer) is present at least at about 99% level, usually at about 99.5% or more.

13. A process of filtration of a composition of crystalline BHA in a liquid when the liquid mixture is:
a. filtered under pressure through an agitated filter, preferably through Nutsche filter or an equivalent equipment, and
b. optionally compressed under pressure into compressed forms comprising one or more of flakes, tablets, granules and the like.

14. BHA in a process of claim 1 to claim 5 and claim 13 compressed form comprising one or more of flakes, tablets, granules and the like prepared by a process of claim 1 to claim 5 and claim 13.

15. BHA in compressed form comprising one or more of flakes, tablets, granules and the like.

16. BHA of claim 15 wherein 3-t-butyl-4-hydroxyanisole (3 isomer) is present at least at about 99% level, usually at about 99.5% or more.

17. BHA of claim 15 wherein TBHQ is present maximum at 100 parts per million level and 3-t-butyl-4-hydroxyanisole (3 isomer) is present at least at about 99% level, usually at about 99.5% or more.
